(19) United States
(12) Patent Application Publication
(10) Pub. No.: US 2024/0249196 A1
(43) Pub. Date: Jul. 25, 2024

(54) DIGITAL SELECTION OF VISCOSITY REDUCING EXCIPIENTS FOR PROTEIN FORMULATIONS

(71) Applicant: MERCK PATENT GMBH, DARMSTADT (DE)

(72) Inventors: Tobias ROSENKRANZ, Darmstadt (DE); Marcel VON DER HAAR, Darmstadt (DE); Adrian SOSIC, Darmstadt (DE); Jan Gerit BRANDENBURG, Darmstadt (DE); Niels BANIK, HEIDELBERG (DE)

(73) Assignee: MERCK PATENT GMBH, DARMSTADT (DE)

(21) Appl. No.: 18/559,906

(22) PCT Filed: May 9, 2022
(86) PCT No.: PCT/EP2022/062389

§ 371 Date: Nov. 9, 2023

(30) Foreign Application Priority Data
May 10, 2021 (EP) 21173067.6

(51) Int. Cl. G06N 20/00

(52) U.S. Cl. CPC G06N 20/00

(57) ABSTRACT

Method for selecting at least one viscosity changing excipient for a formulation containing at least one unknown protein via a computer includes providing a data set that describes the viscosity of several known formulations containing at least one protein and optionally at least one viscosity changing excipient; generating representations of at least one excipient; using a Machine Learning Model executed on the computer to recognize patterns in the data set to evaluate the viscosity changing effect of the viscosity changing excipient to a new formulation containing at least one unknown protein by applying the recognized patterns on provided data of the unknown protein; selecting the at least one excipient according to an acquisition criterion and applying the excipient to the unknown protein, wherein the provided data of the at least one unknown protein describe the viscosity of a protein composition containing the at least one unknown protein.

The present invention relates to a method for selecting viscosity reducing excipients for protein compositions via a computer.

### BACKGROUND AND DESCRIPTION OF THE PRIOR ART

Monoclonal antibodies (mAB) and other protein therapeutics are usually administered parenterally. Subcutaneous injection is particularly popular for the delivery of protein therapeutics due to its potential to simplify patient administration (fast, low-volume injection) and reduce treatment costs (shorter medical assistance). To ensure patient compliance, it is desirable that subcutaneous injection dosage forms be isotonic and can be injected in small volumes (<2.0 ml per injection site). To reduce the injection volume, proteins are often administered with a concentration of 1 mg/ml to 150 mg/ml.

At the same time, mAB-based therapies usually require several mg/kg dosing. The combination of high therapeutic dose and low injection volume thus leads to a need for highly concentrated formulations of therapeutic antibodies. However, being large proteins, antibodies possess a multitude of functional groups in addition to a complex three-dimensional structure. This makes their formulation difficult, particularly when a high concentration is required.

One of the main problems with high concentration protein solutions is viscosity. At high concentrations, proteins tend to form highly viscous solutions largely due to non-native self-association. Additionally, proteins show an increased rate of aggregation and particle formation at such high concentrations.

These problems concern both the manufacturing process and the administration to the patient. In the manufacturing process, highly concentrated protein formulations that are highly viscous present particular difficulties for ultrafiltration and sterile filtration. In addition, tangential flow filtration is often used for the buffer exchange and for the increase of protein concentration. However, because viscous solutions show an increased back pressure and shear stress during injection and filtration, the therapeutic protein is potentially destabilized and/or process times are prolonged. Said increased shear stress frequently results in a loss of product. Both aspects adversely affect process economics.

At the same time, high viscosity is unacceptable when it comes to administration as it significantly limits the injectability of the protein.

Specific excipients and excipient combinations have been identified to reduce viscosity of protein formulations. However, the use of a screening approach to identify the best excipients or excipient combination is time-consuming. In particular in the light of competitive timelines and limited amounts of testing materials (protein), a data-driven approach to reduce the number of experiments and accelerate excipient selection is highly beneficial.

In the state of the art, several approaches for protein formulation design, including Machine Learning models, are known. For instance, the article “Machine learning models of antibody-excipient preferential interactions for use in computational formulation design” (Mol. Pharmaceutics 2020, 17, 3589-3599, DOI. 10.1021/acs.molpharmaceut.0c00629) discloses the ability to describe interactions of formulation excipients with proteins in solution using computer simulations. This enables the formulation design to begin early in the development of a new antibody therapeutic. To do so, it discloses a feature set to numerically describe local regions of an antibody's surface for use in machine learning applications.

Another approach is summarized in the Review “Prediction Machines: Applied Machine Learning for Therapeutic Protein Design and Development” published in the Journal of Pharmaceutical Sciences in December 2nd in 2020. It describes the application of Machine Learning models to better understand the nonlinear concentration-dependent viscosity of protein solutions, predict protein oxidation and deamidation rates, classify sub-visible particles and compare the physical stability of proteins. It further presents improved modelling results for regression and classification of previously published data using various Machine Learning approaches.

Another approach is known from the International Patent application WO 2021/0413S4 A1, which discloses a method for predicting a property of potential protein formulations, where a set of formulation descriptors is classified as belonging to a specific one of a plurality of predetermined groups that each correspond to a different value range for said protein formulation property; classifying the set of descriptors includes applying at least a first portion of the set of descriptors as inputs to a first machine learning model. The method also includes selecting, based on the classification, a second machine learning model from among multiple models corresponding to different groups. The method also includes predicting a value of the protein formulation property that corresponds to the set of descriptors, by applying at least a second portion of the set of formulation descriptors as inputs to the selected model. The method also includes causing the value of the protein formulation property to be displayed to a user and/or stored in a memory.

However, while those publications show a way how to simulate the viscosity of a protein solution, they still have some important shortcomings. First, they all require prior knowledge about the target protein, be it either in the form of data, descriptors, properties or structural details. This is a significant disadvantage because protein developers reluctantly share detailed information about their protein candidates or those information are simply not available. Experiments to obtain these parameters can be cumbersome, time consuming and therefore cost intensive. Furthermore, gathering protein descriptors is complex and might not reflect the protein characteristics well. Regarding the task of formulation design, the most severe shortcoming of the existing approaches is that they are restricted to predicting the concentration-dependent viscosity of a fixed protein formulation and offer no experimental designs that assist the user in exploring other formulations. Therefore, the current state of the art provides no direct way to use the insights gained from its predictions for optimizing formulations involving other excipients, let alone other proteins. Another problem is that these documents only consider the use of single excipients. Combining excipients is beneficial as different excipients might show a synergistic viscosity reduction and/or an improved protein stability compared to a single excipients with similar viscosity reducing effect. Combinations of excipients are not covered by the models of the above mentioned references.

The task of this patent application is to solve the above mentioned problems. It is a further task to find a more efficient Machine Learning based approach for determining and identifying the best excipients or excipient combinations to change the viscosity of protein formulations. It is a further task to find a Machine Learning approach to identify the best experimental design to find an optimal excipient or excipient combination to change the viscosity of protein formulations.

### SUMMARY OF THE INVENTION

The task has been solved by a method for selecting at least one viscosity changing excipient for a formulation containing at least one unknown protein via a computer comprising the following steps of: Providing a data set from a database that describes the viscosity of several known formulations containing at least one protein and optionally at least one viscosity changing excipient; Generating representations of at least one excipient from a list of excipients by the computer via In-Silico-simulations; Using a Machine Learning Model executed on the computer that uses the generated representations of at least one excipient to recognize patterns in the data set to evaluate the viscosity changing effect of at least one viscosity changing excipient chosen from the list of excipients to a new formulation containing at least one unknown protein and the at least one viscosity changing excipient by applying the recognized patterns on provided data of the at least one unknown protein; Selecting, depending on the evaluation result, the at least one excipient from the list according to an acquisition criterion and applying it to the unknown protein, wherein the provided data of the at least one unknown protein are data describing the viscosity of a protein composition containing the at least one unknown protein and optionally with at least one viscosity changing excipient.

This procedure provides a more efficient way to explore the viscosities of protein-excipient formulations compared to state-of-the-art approaches. Main advantage is that, by using the trained Machine Learning Model, the number of real laboratory tests that need to be conducted in order to determine respectively acknowledge the resulting viscosity reached by the used excipients can be reduced significantly. To do so, the used Model evaluates the viscosity changing effect of at least one viscosity changing excipient being added from the list of excipients from the data set which is used for creating the representations. One option of evaluation is that the model predicts the resulting viscosity for all possible excipients (excipients used for the dataset) so that the most suitable combination of unknown protein and excipient(s) can be chosen, but it is also possible that the model predicts the resulting viscosity for a selection of excipients from the list or uses other ways of evaluating. The higher the predictive accuracy of the model, the fewer real, time and resource consuming tests need to be performed. The predictive accuracy of the model generally increases with the number of viscosity measurements provided. If no or only few measurements are available, the Machine Learning Model primarily uses the excipient representations and the data of other known formulations, preferably those that are similar to the new formulation. After the Machine Learning Model has predicted the viscosity of the new formulation and the most suitable excipient(s) have been chosen, the ground truth viscosity of the formulation can be measured and the respective data can be fed back to the model, which is used to enhance the accuracy of subsequent predictions. To run the Machine Learning Model, every standard personal or industrial computer with a processor and respective work and storage memory can be used. It is possible to use the same computer for the In-Silico-simulations and the execution of the Machine Learning Model, but in most cases it is more efficient to use two different computers that are specifically configured to perform the respective application. The criterion for the sufficiency can, for example, suggest excipients that are expected to reduce the viscosity the most or such that yield the largest information gain. The solution of the task also comprises of a software product that is stored on a computer-readable storage medium and comprises instructions which, when executed by a computer, cause the computer to carry out the method steps as disclosed in the previous chapters.

As defined herein, “unknown protein” means proteins that are to be tested by the described method. For those proteins, properties and/or characteristics, like specific protein descriptors, are not necessarily known at the time when the disclosed method is performed. In particular, unknown proteins are proteins that are not in the database of the method as described above. More particularly no comprehensive viscosity measurements with or without viscosity changing excipients are available except for the data describing the viscosity of a protein composition containing the at least one unknown protein and optionally with at least one viscosity changing excipient, which are needed as provided data for the method as described above. It is indeed one of the advantages of the disclosed method against the known prior art that no specific information about the used protein needs to be known and consequently also unknown proteins can be used. The opposite are the “known formulations containing at least one protein and optionally at least one viscosity changing excipient”, which refer to protein compositions, wherein the protein itself and some or all of its properties and/or characteristics are known. In particular, those proteins are in the database, more particularly, viscosity measurements with or without viscosity changing excipients are available. Optionally the formulation can contain one or more known viscosity changing excipients.

As defined herein, “new formulation containing at least one unknown protein and at least one viscosity changing excipient” refers to protein compositions containing a unknown protein as defined above and at least one viscosity changing excipient. The formulation contains one or more known viscosity changing excipients. According to the invention, the viscosity of the new formulations as defined above are predicted. Preferably, the viscosities of more than one formulations are predicted, e.g. a formulation containing at least one unknown protein and at least one viscosity changing excipient A and a formulation containing at least one unknown protein and at least one viscosity changing excipient B. More preferably, the viscosities of all possible formulations are predicted. In this context “all possible combinations” mean all combinations of the at least one unknown protein with at least one viscosity changing excipient chosen from the list of excipients used for generating the data set. In a further embodiment, a group of at least one viscosity changing excipients is chosen from the list of excipients.

As defined herein, “provided data of the at least one unknown protein” are data describing the viscosity of a protein composition containing the at least one unknown protein without a viscosity changing excipient or with at least one viscosity changing excipient. In this context “data describing the viscosity” mean data which is created by at least one viscosity measurement of the protein composition. The protein composition contains at least one unknown protein, wherein the protein itself and its properties and characteristics are unknown. Optionally the protein composition can contain one or more known viscosity changing excipients. The one or more known viscosity changing excipients are viscosity changing excipients that were also used for generating the data set.

The provided data of the at least one unknown proteins do not refer to descriptors or properties of the protein. Gathering of protein descriptors, as used in other methods, is complex and might not reflect the protein characteristics well.

Additionally, there is no need for protein developers to share sensitive information about their protein candidate. It also avoids cost-intensive MD simulations or homology modelling. Furthermore, structural details of the protein, as is required for the other methods, might not be available. In a preferred embodiment of the invention a limited set of viscosity measurements, only one viscosity measurement or no viscosity measurements are needed.

Advantageous and therefore preferred further developments of this invention emerge from the associated subclaims and from the description and the associated drawings.

One of those preferred further developments of the disclosed method comprises that the data set has been generated by experimental measurements and is stored in the database via the computer. The real laboratory tests are also the preferred way to generate the data set that is later used by the Machine Learning Model. The more accurate and representative this data set is, the better are the results of the Machine Learning Model. This point accounts for both the data set containing the viscosity and excipients of the known formulations, and the data set of a new formulation.

Another one of those preferred further developments of the disclosed method comprises that as at least one excipient from the list which changes the viscosity of the new formulation (8) the most sufficient a combination of two or more excipients from the list is used.

Combinations of two or more excipients can be beneficial as different excipients mights shown a synergistic viscosity reduction and/or an improved protein stability compared to a single excipients with similar viscosity reducing effect.

Another one of those preferred further developments of the disclosed method comprises that specific experimental measurements are proposed to a formulation specialist, who conducts these respective experiments in a lab to validate the predicted viscosities and trains the Machine Learning Model with the validated results by adding them to the provided data set in the database via the computer. Additionally, the predicted viscosity values from the Machine Learning Model can also be proposed to the formulation specialist. The mentioned measurements of the resulting viscosity in the new formulation are preferably executed by a formulation specialist. It is possible that the specialist is supported by robotic machinery and software to perform the measurements. If suitable hard- and software is available, the measurements can be performed completely automatically as well.

Another one of those preferred further developments of the disclosed method comprises that initial data describing the viscosity of the new formulation without excipients and/or already validated excipients is used as provided data of the new formulation data. More specifically, if there is already some data known about the new formulation, e.g. from previous measurements or any other source, this data is provided to the Machine Learning Model, which further reduces the amount of tests or measurements necessary to achieve an accurate prediction.

Another one of those preferred further developments of the disclosed method comprises that the Machine Learning Model is created and trained by combining the data set describing the viscosity of at least one prototypical protein formulation with the representations of at least one excipient or a combination thereof. The data set used to create the Machine Learning Model in the first place is the one that comprises of known formulations and their excipients. If the Machine Learning Model is then used to predict viscosity for a new, maybe unknown formulation, it is furthermore trained by either already known characteristics of that formulation with or without excipients, if available, and/or by feeding it with the experimental measurement data resulting from the confirming lab test. If the already known characteristics are not available in the required digital representation form, they need to be converted respectively.

Another one of those preferred further developments of the disclosed method comprises that the viscosity values of a given protein formulation are modelled in the form of a Gaussian process and the model predictions are used to guide the formulation specialist by means of a Bayesian optimal experimental design. With that guide, the formulation specialist can then perform the necessary measurements for the excipient or combination thereof suggested by the Machine Learning Model.

Another one of those preferred further developments of the disclosed method comprise that the training of the Machine Learning Model on the computer is done by performing at least once the following steps of: optimizing the Machine Learning Model Parameters with training data from the data set by maximizing the marginal likelihood of the training data; evaluating the posterior distribution of viscosity values for untested excipients or a combination thereof based on the Machine Learning Model and thereby predicting a viscosity; selecting a new set of excipients or a combination thereof by optimizing an acquisition score obtained from the computed posterior distribution; proposing the new set of excipients or the combination thereof to the formulation specialist, who then conducts the respective experiments in the lab to determine the resulting viscosities; and adding the obtained measurements to the training data.

Another one of those preferred further developments of the disclosed method comprises that the prediction of the viscosity obtained from the posterior distribution of viscosity values is based on a pH-dependent feature vector characterizing the excipients used in the considered formulation and on the used excipient concentration levels. This represents the most preferred way how the Machine Learning Model predicts the viscosity. The Machine Learning Model is, however, not limited to this approach. If there are alternative ways to predict the viscosity value, they can be implemented into and performed by the Machine Learning Model.

Another one of those preferred further developments of the disclosed method comprises that the acquisition criterion assesses which viscosity changing excipient expectedly reduces the viscosity the most. Alternative embodiments may include other acquisition criteria suggesting experiments that, for instance, expectedly yield the largest information gain, result in the largest model change, offer the largest probability of improving the formulation viscosity beyond the level of the best observed setting, yield the largest expected improvement over the current optimal formulation, or that provide any other systematic trade-off between exploration of the formulation search space and exploitation of the knowledge gathered so far.

Another one of those preferred further developments of the disclosed method comprises that the viscosity is measured in a protein formulation containing at least a protein, at least one viscosity changing agent, at least one buffering agent, at least one stabilizer and at least one surfactant in aqueous solution. This combination of components is the most common one and therefore preferably used. However, if there are other combinations required and/or more suitable for the claimed method, they can be used as well.

Another one of those preferred further developments of the disclosed method comprises that the representations of excipients are generated by the computer in the form of physical parameters as well as molecular fingerprints. The physical parameters describe the excipients and its properties so that the Machine Learning Model can process the parameters and use them to predict the viscosity they will cause in a specific protein formulation. Possible parameters include but are not limited to the charge distribution, dipole moment, quadrupole moment trace and anisotropy, polarizability, molecular London dispersion coefficient (C6), logP water/hexane distribution coefficient, solvent accessible surface area, molecular orbital energy HOMO-LUMO gap.

Another solution to the task of this patent application is a Machine Learning Model executed on a computer, which is created and trained as described in the previous chapters.

Another one of those preferred further developments of the disclosed Machine Learning Model comprises that the Gaussian Process is replaced with any other model architecture fulfilling the same purpose, in particular other types of stochastic processes, generalized linear models, neural networks, support vector machines, tree-based models, ensemble models, etc.

### DETAILED DESCRIPTION OF THE INVENTION

The Method, Machine Learning Model and Software Product according to the invention and functionally advantageous developments of those are described in more detail below with reference to the associated drawings using at least one preferred exemplary embodiment. In the drawings, elements that correspond to one another are provided with the same reference numerals.

The drawings show:

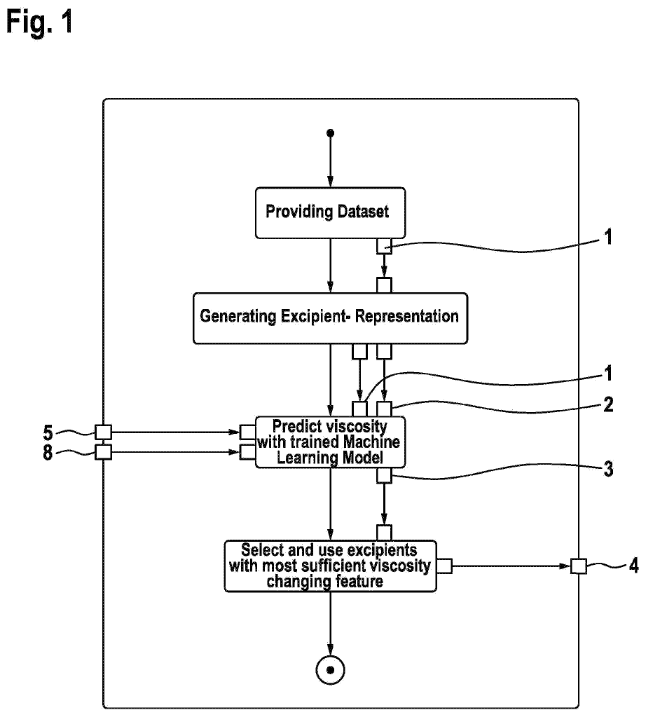

FIG. 1: A process overview about the invented method.

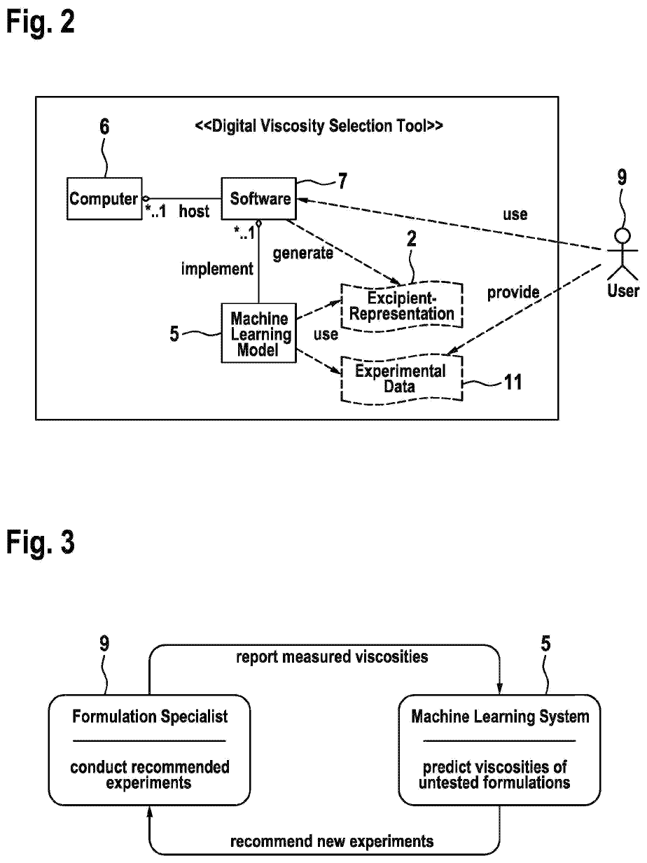

FIG. 2: A summary of the involved system components.

FIG. 3: The training of the used Machine Learning Model.

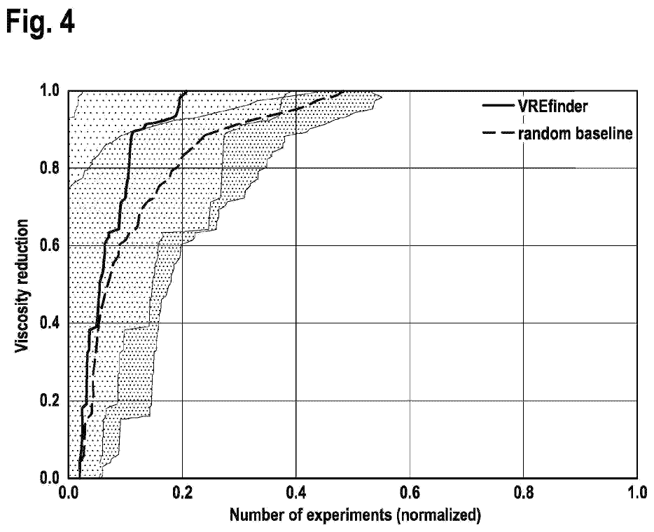

FIG. 4: A result chart showing the performance of the invented method.

The solution to the problem is a software tool enabling a user in data-driven decision making to solve the formulation challenge. The tool consists of three components:
1. Experimental Data 10: The viscosity of various prototypical protein formulations have been measured, generating a data set 1 of 600 data points.
2. Representations of excipients 2 in the form of relevant physical parameters as well as molecular fingerprints. Those are generated via In-Silico-simulations and cross-validated experimentally.
3. A Machine Learning Model 5 that uses the representations 2 from step 2 to recognize patterns in the data from step 1 and predicts viscosities 3 of new protein-excipient formulations 8.

The intended interaction with the developed software tool 7 is described schematically in FIG. 1. FIG. 2 shows an overview about the participating hardware. Apart from the necessary laboratory equipment, the hardware consists mainly of a suitable computer 6 hosting the software 7 that operates the used Machine Learning Model 5. Every kind of computer 6 that is suitable to be used with the respective software 7 can be used, e.g. a standard personal computer or an industrial pc.

The data set 1 is generated by measuring the viscosity of a solution/formulation 8 containing a protein and the viscosity of a solution containing the same protein solutions and additionally containing at least one viscosity reducing excipient 2. Preferably the at least one viscosity reducing excipient 2 is a single viscosity reducing excipient or a combination of two viscosity reducing excipients.

For the measurement of the viscosity reduction, the viscosity of protein compositions not containing a viscosity reducing excipient 2 or a viscosity reducing excipient combination are compared with the viscosity of the protein composition containing a viscosity reducing excipient or a viscosity reducing excipient combination.

The measurements are performed with different proteins at defined concentrations. Different viscosity reducing excipients 2 or viscosity reducing excipient combinations at a defined concentration are used.

Usually, the protein compositions are liquid compositions and additionally contain at least one buffering agent and at least one stabilizer. The buffer and pH is selected depending on the protein and the pH is usually adjusted using NaOH or HCl. The compositions may additionally comprise pharmaceutically acceptable diluents, solvents, carriers, adhesives, binders, preservatives, solubilizers, stabilizer, surfactants, penetration enhancers, emulsifiers or bioavailability enhancers. The skilled person 9 knows how to choose suitable additives and parameters for liquid compositions.

In a preferred embodiment, the compositions according to the invention are liquid formulations 8 and the protein is a therapeutic protein.

Therapeutic proteins encompass antibody-based drugs, Fc fusion proteins, anticoagulants, blood factors, bone morphogenetic proteins, engineered protein scaffolds, enzymes, growth factors, hormones, interferons, interleukins, antibody drug conjugates (ADCs) and thrombolytics. Therapeutic proteins can be naturally occurring proteins or recombinant proteins. Their sequence can be natural or engineered.

In a particularly preferred embodiment, the protein in the compositions and formulations according to the invention is an antibody, in particular a therapeutic antibody.

In a further particularly preferred embodiment, the protein in the compositions and formulations according to the invention is a plasma derived protein, in particular IgG or hyperIgG. Some pharmaceutical formulations containing plasma proteins comprise of mixtures of different plasma proteins.

The term “plasma derived proteins” herein refers to a protein derived from the blood plasma of a donor by plasma fractionation. Said donor can be human or non-human. One example for plasma proteins are immune globulines.

The term “IgG” herein refers to an Immune globbuline type G. The term “IgM” herein refers to an Immune globbuline type M. The term “IgA” herein refers to an Immune globbuline type A.

The term “hyper-IgG” herein refers to a formulation of IgGs purified from a donor that has been infected by or vaccinated against a specific disease. Said donor can be human or non-human.

The term “antibody” herein refers to monoclonal antibodies (including full length or intact monoclonal antibodies), polyclonal antibodies, multivalent antibodies, multispecific antibodies (e.g., bispecific antibodies), and antibody fragments.

Antibody fragments comprise only a portion of an intact antibody, generally including an antigen binding site of the intact antibody and thus retaining the ability to bind antigen. Examples of antibody fragments encompassed by the present definition include: Fab fragments, Fab′ fragments, Fd fragments, Fd′ fragments, Fv fragments, dAb fragments, isolated CDR regions, F(ab′)2 fragments as well as single chain antibody molecules, diabodies and linear antibodies.

In one embodiment, the protein is a biosimilar. A “biosimilar” is herein defined as a biological medicine that is highly similar to another already approved biological medicine. In a preferred embodiment, the biosimilar is a monoclonal antibody.

In one embodiment, the compositions and formulations according to the invention comprise more than one protein species.

The invention is not limited to proteins of a particular molecular weight range. Preferably the protein molecular weight is between 120 kDa and 250 kDa, preferably between 130 kDa and 180 kDa.

One or more protein concentrations are chosen that increase the viscosity of the solution 8 in order to test the viscosity reduction by the viscosity reducing excipients 2. The viscosity of the resultion solution 8 should have a viscosity of at least 20 to 25 mPas⁻¹. In a preferred embodiment, the protein concentration in the compositions and formulations according to the invention is at least 1 mg/ml, at least 50 mg/ml, preferably at least 75 mg/ml and more preferably at least 100 mg/ml. In another preferred embodiment, the protein concentration is between 90 mg/ml and 300 mg/ml, more preferably the protein concentration is between 100 and 250 mg/ml, even more preferable between 120 and 210 mg/ml. The present invention is particularly useful for these high-concentration protein compositions.

There are no limitations for selecting proteins for generating the data set. For example, the following proteins can be used to set up the data set: Cetuximab, Evolocumab, Infliximab, Reslizumab, Etanercept (fusion protein).

As defined herein, “viscosity” refers to the resistance of a substance (typically a liquid) to flow. Viscosity is related to the concept of shear force; it can be understood as the effect of different layers of the fluid exerting shearing force on each other, or on other surfaces, as they move against each other. There are several ways to express viscosity. The units of viscosity are Ns/m², known as Pascal-seconds (Pas). Viscosity can be “kinematic” or “absolute”. Kinematic viscosity is a measure of the rate at which momentum is transferred through a fluid. It is measured in Stokes (St). The kinematic viscosity is a measure of the resistive flow of a fluid under the influence of gravity. When two fluids of equal volume and differing viscosity are placed in identical capillary viscometers and allowed to flow by gravity, the more viscous fluid takes longer than the less viscous fluid to flow through the capillary. If, for example, one fluid takes 200 seconds (s) to complete its flow and another fluid takes 400 s, the second fluid is called twice as viscous as the first on a kinematic viscosity scale. The dimension of kinematic viscosity is length2/time. Commonly, kinematic viscosity is expressed in centiStokes (cSt). The SI unit of kinematic viscosity is mm²/s, which is equal to 1 cSt. The “absolute viscosity,” sometimes called “dynamic viscosity” or “simple viscosity,” is the product of kinematic viscosity and fluid density. Absolute viscosity is expressed in units of centipoise (cP). The SI unit of absolute viscosity is the milliPascal-second (mPas), where 1 cP=1 mPas.

Viscosity may be measured by using, for example, a viscometer at a given shear rate or multiple shear rates. An “extrapolated zero-shear” viscosity can be determined by creating a best fit line of the four highest-shear points on a plot of absolute viscosity versus shear rate, and linearly extrapolating viscosity back to zero-shear. Alternatively, for a Newtonian fluid, viscosity can be determined by averaging viscosity values at multiple shear rates. Viscosity can also be measured using a microfluidic viscometer at single or multiple shear rates (also called flow rates), wherein absolute viscosity is derived from a change in pressure as a liquid flows through a channel. Viscosity equals shear stress over shear rate. Viscosities measured with microfluidic viscometers can, in some embodiments, be directly compared to extrapolated zero-shear viscosities, for example those extrapolated from viscosities measured at multiple shear rates using a cone and plate viscometer. According to the invention, viscosity of compositions and formulations 8 is reduced when at least one of the methods described above show a stabilizing effect. Preferably, viscosity is measured at 20° C. using mVROC™ Technology. More preferably the viscosity is measured using mVROC™ Technology at 20° C. Most preferably the viscosity is measured at 20° C. using mVROC™ Technology and using a 500 μl syringe, a shear rate of 3000 s⁻¹ or 2000 s⁻¹ and a volume of 200 μl. The person ordinary skilled in the art is familiar with the viscosity measurement using mVROC™ Technology, especially with selecting the parameters described above. Detailed specifications, methods and setting can be found in the 901003.5.1-mVROC_User's_Manual.

“Shear rate” herein refers to the rate of change of velocity at which one layer of fluid passes over an adjacent layer. The velocity gradient is the rate of change of velocity with distance from the plates. This simple case shows the uniform velocity gradient with shear rate (v1−v2)/h in units of (cm/sec)/(cm)=1/sec. Hence, shear rate units are reciprocal seconds or, in general, reciprocal time. For a microfluidic viscometer, change in pressure and flow rate are related to shear rate. “Shear rate” is to the speed with which a material is deformed. Formulations 8 containing proteins and viscosity-lowering agents are typically measured at shear rates ranging from about 0.5 s⁻¹ to about 200 s⁻¹ when measured using a cone and plate viscometer and a spindle appropriately chosen by one skilled in the art to accurately measure viscosities in the viscosity range of the sample of interest (i.e., a sample of 20 cP is most accurately measured on a CPE40 spindle affixed to a DV2T viscometer (Brookfield)); greater than about 20 s⁻¹ to about 3,000 s⁻¹ when measured using a microfluidic viscometer.

For classical “Newtonian” fluids, as generally used herein, viscosity is essentially independent of shear rate. For “non-Newtonian fluids,” however, viscosity either decreases or increases with increasing shear rate, e.g., the fluids are “shear thinning” or “shear thickening”, respectively. In the case of concentrated (i.e., high-concentration) protein solutions, this may manifest as pseudoplastic shear-thinning behavior, i.e., a decrease in viscosity with shear rate.

In one embodiment, the compositions and formulations of the invention show a reduction of viscosity of at least 5%, 10%, 15%, 20%, 25%, 30%, 35%, 40%, 45%, 50%, 55%, 60%, 65%, 70% or 75% compared to an identical composition not comprising the at least one first excipient.

In one embodiment, the compositions and formulations of the invention show a reduction of viscosity of at least 5%, 10%, 15%, 20%, 25%, 30%, 35%, 40%, 45%, 50%, 55%, 60%, 65%, 70% or 75% compared to an identical composition not comprising the at least one first and at least one second excipient 2.

The invention further provides a pharmaceutical formulation 8 according to the invention whereas the viscosity is between 1 mPas and 60 mPas, preferably between 1 mPas and 50 mPas, more preferably between 1 mPas and 30 mPas, most preferably between 1 mPas and 20 mPas.

The compositions usually have a pH between 4 and 8, preferably between 5 and 7.2. In one embodiment, the compositions and formulations 8 have a pH of exactly 5 or exactly 7.2. The pH is selected depending on the protein and the pH is usually adjusted using NaOH or HCl. The skilled person knows how to choose a pH for protein compositions.

The at least one stabilizer is a compound that is suitable to increase the stability of a protein. Suitable stabilizer are known in the art and include suitable sugars and/or a surfactants. Suitable sugars as stabilizers are known in the literature, e.g. sucrose or trehalose. In a preferred embodiment, the sugar is sucrose. Suitable surfactants are known in the literature, e.g. polysorbate 20 or polysorbate 80 or poloxamer 188. In another preferred embodiment, the surfactant is polysorbate 80. The addition of a further stabilizers additionally enhances the stabilizing effect of the compositions according to the inventions. Preferably the sugar has a concentration of 50 to 100 mg/ml, more preferably 50 mg/ml sucrose. Preferably the surfactant has a concentration of 0.01 to 0.2 mg/ml, more preferably 0.05 mg/ml of polysorbate 80.

The at least one buffering agent that is suitable for protein solutions is added to prepare a buffer solution. Suitable buffers are known in the art, e.g. an acetate- citrate- or phosphate salt phosphate buffer. The buffer usually has a concentration of 1 to 50 mM.

According to the invention, a “viscosity changing excipient 2” is a compound that can influence the viscosity of a liquid formulation. This definition includes “viscosity reducing excipients 2” that are suitable to reduce viscosity of a liquid formulation 8 when added to the formulation 8 in a concentration range as defined below. Preferably the liquid formulation 8 is a protein solution.

There are no limitations for selecting viscosity reducing excipients 2 for generating the data set 1. The following viscosity reducing excipients 2 can be used to set up the data set: Guanidine hydrochloride, L-Arginine, L-Carnithine hydrochloride, L-Ornithine hydrochloride, L-Serine, Lysine, Meglumine, Quinine hydrochloride, Thiamine hydrochloride, Ascorbic acid, Benzenesulfonic acid, Camphorsulfonic acid, Thiamine pyrophosphate, Di-Sodium Succinate, di-Sodium Tartrate, Folic acid, Gluconic acid, Glucuronic acid, Pyridoxin, Sodium-p-toluenesulfonate, Thiamine monophosphate, Urea, Aminocaproic acid, Caffeine, Cyanocobalamin, Glycine, Isoleucine, Leucine, Nicotinamide, Phenylalanine, Proline, Sodium Chloride, Valine.

Such viscosity reducing agents are used in a concentration that is suitable to reduce the viscosity of the protein solution 8. In a preferred embodiment, the protein concentration in the compositions and formulations according to the invention is at least 1 mg/ml, at least 50 mg/ml, preferably at least 75 mg/ml and more preferably at least 100 mg/ml. In another preferred embodiment, the protein concentration is between 90 mg/ml and 300 mg/ml, more preferably the protein concentration is between 100 and 250 mg/ml, even more preferable between 120 and 210 mg/ml. Preferably one viscosity changing excipient 2 is used at a concentration of at maximum 200 mM, more preferably at maximum 150 mM, most preferably at a concentration of 75 mM or 150 mM. In case two viscosity changing excipients 2 are used, preferably each is at a concentration of at maximum 150 mM, more preferably each at maximum 100 mM, most preferably each at a concentration of 75 mM. As a resulting concentration level of more than 300 mM is not preferable, the concentration for both excipients 2 should not exceed 150 mM. If there is, for any reason, an uneven distribution between the two excipients 2 preferred, the ratio changes respectively. The same rules account if more than two excipients 2 are used. Those levels of concentration are the most effective for reducing the viscosity and are, therefore, preferred. However, the method is not limited to theses specific values.

The excipient data set 1 is based on simplified molecular-input line-entry system (SMILES) representations. Every viscosity measurement is performed at defined, but different environments characterized by their pH value. To incorporate changes in excipient protonation, a pH-dependent microspecies distribution is generated using ChemAxons predictor. The pH-dependence is between pH 4-pH 8. Each microspecies is converted into a three-dimensional structure using a Marvin molconverter. From the three dimensional trial-structure an ensemble of all conformers in water solution populated at room temperature is computed. For this, the CREST algorithm is employed, which is a meta-dynamics, structure-crossing, simulated annealing based global search running on a quantum mechanical potential energy surface at the extended tight-binding level including a generalized born and surface accessible implicit area solvation model (GFN2-xTB+GBSA(water)). Zero-point and thermodynamic contributions are included via a rigid-rotor-harmonic-oscillator (RRHO) model. The individual geometries have been further refined with the density functional approximation B97-3c within a conductor like screening model for real solvents (COSMO-RS) within its 2019.0.4 parametrization. Final single-points used for the Boltzmann population of the conformer ensemble are composed of the electronic energy, the RRHO contribution and the solvation free energy. Structures with contribution below 1% are disregarded.

Those microspecies ensembles are the basis for quantum chemical calculations at the density functional theory level to simulate molecular observables, like charge distribution, dipole moment, quadrupole moment trace and anisotropy, polarizability, molecular London dispersion coefficient (C6), logP water/hexane distribution coefficient, solvent accessible surface area, molecular orbital energy HOMO-LUMO gap. These quantum mechanic features are complemented with a set of topological molecular fingerprints. This augmentation set of 200 standardized fingerprints is generated based on the same microspecies ensemble using RDKit.

Together, this yields for every single excipient 2 a high-dimensional pH-dependent feature vector.

The developed Machine Learning Model 5 combines the experimental data obtained in the laboratory with the computed In-Silico excipient features to build a predictive model of formulation viscosities. Based on this model 5, an optimal experimentation schedule is provided. The formulation specialist 9 uses these suggestions and performs the recommended experiments 10 and subsequently feeds the newly obtained viscosity data into the system, as FIG. 3 shows exemplarily. Through this process, the execution of experiments is focused on formulations that have the highest likelihood of viscosity reduction.

For a given protein, viscosities values are modelled in the form of a Gaussian process (GP), and the predictions of the model 5 are used in a preferred embodiment to guide the formulation specialist 9 by means of Bayesian optimal experimental design. This guidance comprises of several steps:
1. Given a possibly empty set of viscosity measurements for certain excipients/excipient combinations (=training data), the GP model parameters are optimized by maximizing the marginal likelihood of the training data.
2. The posterior distribution of viscosity values for untested excipients/excipient combinations are evaluated based on the GP model.
3. A new set of excipient/excipient combinations is selected by optimizing an acquisition score obtained from the computed posterior distribution.
4. The new set of excipients/excipient combinations is proposed to the formulation specialist 9, who then conducts the respective experiments in the lab to determine the resulting viscosities.
5. The obtained measurements are added to the training data and the process repeats from Step 1.

The prediction in Step 2 is based on the pH-dependent feature vector characterizing the excipients 2 used in the considered formulation 8, as well as on the excipient concentration levels.

A challenge of this procedure in Steps 1 to 5 is that the measured viscosities not only depend on the chosen excipient combination but also on the ground truth protein concentration, which can vary in each measurement. To account for deviations from the target concentration, the GP model 5 is designed to predict relative changes in viscosity rather than absolute viscosity values. More precisely, it predicts the relative viscosity reduction with respect to the theoretical viscosity level that would be achieved at the actual protein concentration without excipients. The required theoretical values are obtained from an exponential regression model computed from concentration-dependent viscosity measurements of the unformulated protein solution.

While the considered design of experiments in Steps 1 to 5 follows a typical optimization procedure, existing black-box GP models 5 provided by leading software suites, such as GPyTorch, BoTorch, GPflow, cannot be applied to the given scenario due to the particular data characteristics that need to be encoded. Therefore, a specialized GP kernel structure has been designed to account for the following domain and problem-specific properties. Those properties are:
The combination of excipients 2 contained in a given formulation has no natural order, i.e. adding ExcipientA+ExcipientB is equivalent to adding ExcipientB+ExcipientA. The used kernel is designed to be permutation-invariant with respect to the added excipients 2.
A given formulation 8 can contain a varying number of excipients 2. The kernel has been constructed to handle flexible excipient numbers.
The evoked viscosity-reducing effect of the formulation 8 depends on both the given protein concentration and the applied excipient concentrations. The dependency on these concentrations is explicitly reflected in the structure of the used kernel.
Combining excipients 2 can result in synergetic viscosity-reducing effects that may not be described through the characteristics of each excipient in isolation. Common generic kernel structures based on automatic relevance detection of individual feature dimensions cannot sufficiently capture these multivariate relationships. In order to generalize from the measurement data to untested excipient combinations, the used kernel uses a linear subspace projection that is optimized during the parameter fitting process.

Particularly challenging is the generalization of viscosity predictions 3 to new proteins. This is due to missing chemical information characterizing global as well as local interactions of the protein. Therefore, a further, extended preferred embodiment is recommended. It comprises of a database containing viscosity measurements of various formulations 8 that constitute prototypical interaction patterns between proteins and excipients 2. These interaction patterns can be used as prior information for the viscosity prediction 3 of new proteins in the form of an additional kernel component that biases the predictions towards those of matching protein-excipient patterns. One approach to achieve this, though others are possible, is by capturing the protein influence via a multi-task kernel model, such as an Intrinsic Coregionalization Model or variants thereof. In alternative embodiments of the invention, where the Gaussian process is replaced with other Machine Learning models, the task of generalizing across proteins can be taken over by other appropriate model components.

In the following, a specific working example is disclosed to demonstrate the advantage of using the software tool 7 compared to performing an uninformed search where the next experiment is selected at random.

The goal is to reduce the viscosity of a protein solution 8 below a specified threshold. Given the vast landscape of excipients 2 available on the market, it is difficult to find a suitable excipient combination. In order to avoid an exhaustive screening study in which all candidate formulations are tested, an informed, data-driven search with the help of the proposed software tool 7 is performed.

To this end, the following steps are executed:
1) Specific formulation conditions, in particular the pH value and which excipients 2 may be considered as potential candidates, are defined.
2) A small number of concentration-dependent viscosity measurements of a solution containing a new protein without the at least one viscosity reducing agent are performed. This data is fed into the software tool 7 to estimate a base viscosity curve for the unformulated protein based on viscosity predictions 3.

By consulting the software tool 7 after each measurement taken, a user is instructed which protein concentration to consider next and gets informed once a sufficient amount of data has been collected.
3) The software tool 7 recommends then a first excipient 2 or excipient combination to be tested. The user conducts the respective experiment 10 in the lab and reports the measured viscosity back to the tool. In an iterative process, the user is prompted to perform further experiments in response to the latest measurements reported to the tool 7, until a formulation 8 with sufficiently low viscosity is found.

If measurements were already taken before using the tool 7, e.g. for excipients 2 that are not on the candidate list, the user can report the corresponding viscosities before initiating the process. That way, the tool 7 can give improved recommendations from the start.

In an alternative embodiment, the user can perform several experiments at once before consulting the software tool 7 after each iteration. In this so called “batch mode”, the user can enter the desired number of experiments to be performed in parallel during the next iteration, e.g. for the purpose of scheduling laboratory resources. The software tool 7 will then optimize its recommendations in such a way as to optimize the expected information gain that results from conducting the experiments simultaneously.

FIG. 4 shows the achieved viscosity reduction of both search strategies over the number of experiments conducted by the user. For the given example, a total of 629 experiments were considered covering 6 proteins and 33 excipients. In order to average the results over all tested proteins, the measured viscosity reductions are reported relative to the maximum observed reduction per protein and the number of experimentation steps is shown relative to the total number of experiments 10 conducted per protein. Depicted are the resulting mean values (solid lines) and standard deviations (shaded area) obtained from several repetitions of the experiment. These repetitions where obtained by considering different sets of initial measurements provided to the software tool 7 and different random experimentation paths for the random baseline strategy.

In agreement with the theoretical number of required steps, the random strategy finds the optimal excipient combination in expectation after conducting 50% of all possible experiments. Using the invented approach, this number can be reduced by half on average.

A further embodiment of the present invention is the new formulation 8 containing at least one viscosity changing excipient 2 selected via the method provided above.

A further embodiment of the present invention is a pharmaceutical formulation containing the new formulation 8 and the at least one viscosity changing excipient 2 selected via the method provided above.

A further embodiment of the present invention is a pharmaceutical formulation containing the new formulation 8 and the at least one viscosity changing excipient 2 selected via the method provided above.

### Examples

### 1. Generating the Experimental Data/Viscosity Measurements

### General Concept of the Experiments

For generating the experimental data for the data set 1, various protein composition have been prepared and the viscosity reduction of different viscosity reducing excipient were tested.

The following commercially available proteins were used: Cetuximab, Evolocumab, Infliximab, Reslizumab, Etanercept.

The following commercially available viscosity reducing excipients 2 were used: Guanidine hydrochloride, L-Arginine, L-Carnithine hydrochloride, L-Ornithine hydrochloride, L-Serine, Lysine, Meglumine, Quinine hydrochloride, Thiamine hydrochloride, Ascorbic acid, Benzenesulfonic acid, Camphorsulfonic acid, Thiamine pyrophosphate, Di-Sodium Succinate, di-Sodium Tartrate, Folic acid, Gluconic acid, Glucuronic acid, Pyridoxin, Sodium-p-toluenesulfonate, Thiamine monophosphate, Urea, Aminocaproic acid, Caffeine, Cyanocobalamin, Glycine, Isoleucine, Leucine, Nicotinamide, Phenylalanine, Proline, Sodium Chloride, Valine and combinations thereof.

In the following, the measurement of the viscosity reduction of Valine as viscosity reducing excipient 2 on an Infliximab solution is exemplified. The general concept of this particular example can be transferred to all other proteins and viscosity reducing agents used.

In case a single viscosity reducing agent was used, the viscosity was generally measured at a concentration of 150 mM. In case a combination of two excipients 2 was used, the viscosity was generally measured at a concentration of 75 mM for each of the excipients. In some instances, the concentration of the viscosity reducing excipient 2 was adjusted according to the solubility of the excipient 2.

Depending on the protein used, the buffer, pH, protein concentrations and optional stabilizers and/or surfactants were selected. Usually buffer, pH, stabilizers and/or surfactants of the commercially available products containing the proteins were used. Proteins solutions were concentrated to yield a solution with a viscosity of at least 20 mPas⁻¹. In some instances, the viscosity was measured at more than one protein concentration.

### Viscosity Measurement

### Buffer Preparation

5 mM phosphate buffer was prepared by appropriately mixing sodium dihydrogenphosphate and di-sodium hydrogenphosphate to yield a pH of 7.2 and dissolving the mixture in ultrapure water. The ratio was determined using the Henderson-Hasselbalch equation. pH was adjusted using HCl and NaOH where necessary. 50 mg/ml sucrose and 0.05 mg/ml polysorbate 80 were added as stabilizers.

### Sample Preparation

Individual excipient solution of 150 mM Valine was prepared in phosphate buffer pH 7.2. The pH was adjusted using HCl or NaOH where necessary.

A concentrated Infliximab solution containing the desired excipients was prepared using centrifugal filters (Amicon, 30 kDa MWCO) to exchange the original buffer with a buffer containing the respective excipient and to reduce the volume of the solution 8. The protein was subsequently diluted to 122 mg/ml and 143 mg/ml, respectively. In an analogous manner, an otherwise identical protein solution not containing Valine was prepared.

### Protein Concentration Measurements

Protein Concentration was determined using absorption spectroscopy applying Lambert-Beer's law. When excipients themselves had a strong absorbance at 280 nm, a Bradford assay was used.

Concentrated protein solutions were diluted so that their expected concentration would lie between 0.3 and 1.0 mg/mL in the measurement.

For absorption spectroscopy, the absorbance at 280 nm was measured using a BioSpectrometer® kinetic (Eppendorf, Hamburg, Germany) with a protein extinction coefficient of A0.1%, 280 nm=1.428.

Some excipients 2 have themselves a strong absorption at 280 nm, which makes it necessary to use a Bradford assay for concentration determination. For the Bradford assay, a kit as well as Bovine Gamma Globulin Standard from Thermo Scientific™ (Thermo Fisher, Waltham, Massachusetts, USA) were used. Absorption was measured at 595 nm using a Multiskan™ Wellplatereader (Thermo Fisher, Waltham, Massachusetts, USA). Protein concentrations were determined by linear regression of a standard curve from 125 to 1500 μg/ml.

### Viscosity Measurements

The mVROC™ Technology (Rheo Sense, San Ramon, California, USA) was used for viscosity measurements.

Measurements were performed at 20° C. using a 500 μl syringe and a shear rate of 3000 s⁻¹. A volume of 200 μl was used. All samples were measured as triplicates. The viscosity reduction was calculated by comparing the absolute viscosity of the protein compositions with and without Valine.

### 2. Concentration-Dependent Viscosity Measurements of the Raw Protein Solution

In the following, the measurement of the concentration-dependent viscosity of Infliximab is exemplified. The general concept of this particular example can be transferred to all other proteins.

Buffer and sample preparation were performed as described above. The protein was subsequently diluted to 13, 30, 42, 68, 79, 80, 103, 110, 117.30, 121 and 148.2 mg/ml, respectively. Protein concentration measurements using absorption spectroscopy applying Lambert-Beer's law were performed as described above. Viscosity measurements of the different Infliximab concentrations using the mVROC™ Technology (RheoSense, San Ramon, California, USA) were performed.

### LIST OF REFERENCES

1 Data set
2 Excipient (representation)
3 Predicted viscosity
4 Chosen excipient (combination)
5 Machine Learning Model
6 Used computer
7 Software Tool
8 New formulation
9 User (formulation specialist)
10 Experimental measurement data
11 Unknown protein

1. A method for selecting at least one viscosity changing excipient (2) for a formulation (8) containing at least one unknown protein (11) via a computer (6) comprising the following steps:
Providing a data set (1) from a database that describes the viscosity of several known formulations containing at least one protein and optionally at least one viscosity changing excipient (2);
Generating representations of at least one excipient (2) from a list of excipients by the computer (6) via In-Silico-simulations;
Using a Machine Learning Model (5) executed on the computer (6) that uses the generated representations of at least one excipient (2) to recognize patterns in the data set (1) to evaluate the viscosity changing effect of at least one viscosity changing excipient (2) chosen from the list of excipients to a new formulation (8) containing at least one unknown protein (11) and the at least one viscosity changing excipient (2) by applying the recognized patterns on provided data of the at least one unknown protein (11);
Selecting, depending on the evaluation result, the at least one excipient from the list according to an acquisition criterion and applying it to the unknown protein (11), wherein the provided data of the at least one unknown protein (11) are data describing the viscosity of a protein composition containing the at least one unknown protein (11) and optionally with at least one viscosity changing excipient (2).

2. The method according to claim 1, wherein the evaluation of the viscosity changing effect of at least one viscosity changing excipient (2) is done by predicting the viscosity (3) of the new formulation (8) containing at least one unknown protein (11) and at least one viscosity changing excipient (2).

3. The method according to claim 1, wherein the data set (1) has been generated by experimental measurements (10) and is stored in the database via the computer (6).

4. The method according to claim 1, wherein as at least one excipient from the list which changes the viscosity of the new formulation (8) the most sufficient a combination of two or more excipients from the list is used.

5. The method according to claim 1, wherein at least one specific experimental measurement (10) is proposed to a formulation specialist (9), who conducts the at least one respective experiment (10) in a lab to validate the predicted viscosities (3) and trains the Machine Learning Model (5) with the validated results by adding them to the provided data set (1) in the database via the computer (6).

6. The method according to claim 1, wherein the Machine Learning Model (5) is created and trained by combining the data set (1) describing the viscosity of at least one prototypical protein formulation (8) with the representations of the at least one viscosity changing excipient (2) or a combination thereof.

7. The method according to claim 6, wherein the viscosity values of a given formulation (8) are modelled via the Machine Learning Model (5) in the form of a Gaussian process and the model predictions (3) are used to guide the formulation specialist (9) by means of a Bayesian optimal experimental design.

8. The method according to claim 6, wherein the training of the Machine Learning Model (5) on the computer (6) is done by performing at least once the following steps:
Optimizing the Machine Learning Model Parameters with training data from the data set (1) by maximizing the marginal likelihood of the training data;
Evaluating a posterior distribution of viscosity values for untested excipients (2) or a combination thereof based on the Machine Learning Model (5) and thereby predicting a viscosity (3);
Selecting a new set of excipients (2) or a combination thereof by optimizing an acquisition score obtained from the computed posterior distribution;
Proposing the new set of excipients (2) or the combination thereof to the formulation specialist (9), who then conducts the respective experiments (10) in the lab to determine the resulting viscosities;
Adding the obtained measurements (10) to the training data.

9. The method according to claim 8, wherein the prediction of the viscosity (3) obtained from the posterior distribution of viscosity values (3) is based on a pH-dependent feature vector characterizing the excipients (2) used in the considered formulation (8) and on the used excipient concentration levels.

10. The method according to claim 1, wherein the acquisition criterion is which viscosity changing excipient (2) reduces the viscosity the most.

11. The method according to claim 1, wherein the representations of excipients (2) are generated by the computer (6) in the form of physical parameters as well as molecular fingerprints.

12. The method according to claim 1, wherein the generated representations of excipients (2) are cross-validated experimentally.

13. The method according to claim 1, wherein the generated representations of excipients (2) include quantum mechanic features, optionally complemented with a set of topological molecular fingerprints.

14. A Machine Learning Model performed on a computer which is created and trained according to claim 5.

15. The Machine Learning Model according to claim 14, wherein the Gaussian Process is replaced with any other model architecture fulfilling the same purpose.

16. The Machine Learning Model according to claim 14, wherein the Gaussian Process is replaced with another type of stochastic process, a deep Bayesian network, a generalized linear model, a neural network, a support vector machine, a tree-based model, or an ensemble model.
